(19) United States
(12) Patent Application Publication
(10) Pub. No.: US 2008/0277606 A1
(43) Pub. Date: Nov. 13, 2008

(54) Microfluidic Device

(75) Inventors: Xuhua Wang, Oxford (GB); Oliver Hofmann, London (GB); John De Mello, Teddington (GB); Andrew De Mello, Middlesex (GB); Donal Bradley, Bucks (GB)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: MOLECULAR VISION LIMITED, London (GB)

(21) Appl. No.: 12/093,268

(22) PCT Filed: Nov. 9, 2006
(86) PCT No.: PCT/GB2006/004198

§ 371 Date: May 9, 2008

(30) Foreign Application Priority Data
Nov. 11, 2005 (GB) 0523079.2
Mar. 9, 2006 (GB) 0604799.7

(51) Int. Cl. A61B 6/00; C09K 11/02

(52) U.S. Cl. 250/581; 252/301.35; 264/21

(57) ABSTRACT

The invention relates to a microfluidic device comprising a zone for receiving a sample, an excitation source and a detector, wherein a portion of the zone allows transmission of energy at a required wavelength range and prevents transmission of energy at all other wavelengths and uses of the device in disposable on-chip fluorescent detection.

The present invention relates to a microfluidic device comprising a monolithically integrated dye-doped polydimethyl siloxane (PDMS) optical filter and its use in disposable on-chip fluorescence detection

Microfluidic devices have had a tremendous impact on the development of miniaturized and micro total analysis systems (μTAS) in chemical engineering and molecular biology. These systems have potential for wide application in clinical point-of-care diagnostics, genetic analysis, drug discovery, food industry, environmental monitoring, forensic investigation and chemical/biological warfare defense. Miniaturization of devices leads to decreased time of analysis, reduced consumption of reagents and analytes, increased separation efficiency, high throughput screening, portability and disposability.

A major goal of microfluidics research is the development of integrated systems that successfully incorporate all stages of a complete chemical or biological analysis into a single device. Typical stages in an analysis include sampling, pre-treatment, chemical reactions, analytical separations, and analyte detection. The final step is often the most challenging due to the small quantities of analyte present and the consequent need for high sensitivity detection. In practice, optical techniques are often the only ones that provide adequate sensitivity, and considerable efforts have therefore been devoted to developing integrated optical components for use in microfluidic devices. In this regard efforts have been made to integrate amongst other things microlenses, filters, mirrors, gratings, waveguides, light sources and photodetectors.

Detection of analytes in micro total analysis systems can be achieved by a number of methods, of which fluorescence based detection is the most widely used. Fluorescence based detection is of particular use for example in the detection of DNA due to its high sensitivity, ease of automation and real time detection. Many current biochemistry protocols, such as Sanger sequencing and the polymerase chain reaction (PCR) have been adapted to fluorescent labelling methods. The incorporation of such detection methods into chip based assays is therefore an important development in the field and fluorescence detection can be used in a variety of applications on an analysis chip.

Fluorescence detection sensitivity is severely compromised by background signals, which may originate from endogenous sample constituents or from unbound or nonspecifically bound probes (reagent background) scattered light from the excitation light source or direct excitation of the detector by the light source. Detection of autofluorescence and scattered or direct excitation light can be minimized by selecting filters to absorb an unwanted signal and transmit a desired signal. In this way, the signal-to-noise ratio can be enhanced greatly. Therefore, the use of filters plays an important role in fluorescence detection.

Conventional fluorescence-sensing systems use bulky and discrete components that are expensive and non-portable. The deployment of such conventional optical systems in combination with a micro total analysis system is therefore inconvenient. Miniaturization of such systems is not straightforward since it involves the design of compact light sources, filters, and sensitive on-chip photodetectors.

Functional integration of optical components within monolithic substrates in the micro total analysis system and lab-on-a-chip field has recently been the subject of significant research and development activity. In fluorescence based detection, optical short pass filters are used for sharpening of the excitation light and long-pass filters are generally employed to prevent excitation light from reaching the detector. Often these filters are used as external components, even in otherwise integrated optical microchips.

In the context of microfluidic devices, optical long-pass filters play a particularly important role. In conventional fluorescence detection, the excitation source and detector are usually arranged orthogonally to one another to prevent direct illumination of the detector by the excitation source. This orthogonal geometry, however, is difficult to implement in a microfluidic environment since it requires the production of optical grade side-surfaces and the non-facile integration of optical components onto the side-surfaces of the microfluidic chip. Light sources and detectors are most conveniently located on the upper and lower faces of the microfluidic chip in a co-linear geometry, but this would ordinarily flood the detector with direct light from the excitation source, masking the typically weak fluorescence signal from the analyte. The key to achieving effective discrimination of the excitation and emission light in this ‘head-on’ configuration is the use of a long-pass filter in front of the detector, which blocks the excitation light and passes only the longer wavelength emission signal. The use of long-pass filters for this purpose is well established but relies on discrete stand-alone filters—an approach which yields satisfactory optical performance but prevents monolithic integration and increases the distance between the microchannel and the detector, leading to inefficient collection of the fluorescence signal.

In assembled hybrid devices, a commercial ZnS/YF₃ interference filter has been used in a backside illumination geometry to block out scattered excitation light. Similarly, commercial long-pass and band-pass filters have been used in the hybrid epi-fluorescence detection module of a hand-held protein analyzer. Combined holographic notch plus and interference filters have been attached to a glass microchip via a PDMS layer. In addition a 80-micrometre-thick yellow polycarbonate long-pass filter sandwiched between the PDMS microfluidic layer and a second PDMS plate comprising a microavalanche photodiode has been employed. However in this assembly, a 5-times higher background signal was still observed with the light source turned on, indicating inefficient blocking. Such hybrid assembly approaches suffer from large distances between microchannel, filter and detector, resulting in inefficient collection of the fluorescent signal and limited detection sensitivity. Furthermore, the background signal obtained in such hybrid assemblies is too high for sensitive detection.

A monolithically integrated multilayer interference filter fabricated on top of a PIN silicon photodiode has been developed for silicon based microchips. The filters typically comprise up to 40 alternating SiO₂/TiO₂ layers and exhibit 5%-transmittance at 490 nm and 90%-transmittance at 510 nm. However, these integrated DNA analysis microdevices are currently unsuitable for disposable diagnostic tests given the complex fabrication and associated high cost.

A CdS thin-film filter has been developed which exhibits strong blocking of the excitation light for most incident angles. However, only ˜40% of the emission signal is transmitted, resulting in low sensitivity. Long-pass filters based on doped AlGaAs or multi-layer Fabry-Perot cavities have also been reported to suffer from side illumination and low transmittance, respectively.

Microfluidic systems can be manufactured in glass, oxidized silicon or in a polymer such as polydimethylsiloxane. Polydimethylsiloxane is particularly favoured for use in microfluidic devices as it allows the reproduction of features on the micron and submicron scale with high fidelity by replica moulding, has good optical transparency down to 280 nm, cures at low temperature, is non-toxic, can be deformed reversibly, seals reversibly to itself and other materials or can seal irreversibly after exposure to air or oxygen plasma by formation of covalent bonds.

The interference-filters and CdS-filters described above could in principle be straightforwardly integrated with glass microfluidic chips, but they are unsuitable for conformable elastomeric materials such as PDMS since polycrystalline materials such as CdS, TiO₂ and SiO₂ are typically deposited at relatively high temperatures (>300° C.) and have a tendency to crack when the substrate is flexed.

Polydimethyl siloxane has previously been modified for biomedical applications by changing its mechanical properties. In addition stained polydimethylsiloxane produced by post-polymerization staining with Sudan Red has been used as a photothermal detector. Self-assembly in mesoscale systems was investigated by post-polymerisation staining of polydimethylsiloxane with perfluorodecalin, perfluoromethyldecalin, crystal violet and Sudan Red 7B. However, such staining processes are diffusion based and thus slow, dye incorporation is often shallow and non-uniform and the dye load is difficult to control.

The use of dye doped polydimethylsiloxane layers as filters have been suggested. However, problems associated with incorporation of the dye into the polydimethylsiloxane have prevented the realisation of this suggestion, and currently only an example of an external unstructured post-polymerisation stained polydimethylsiloxane layer for use as a filter has been disclosed.

There is a need in the art for an improved microfluidic device for use in optically based detection system which allow sensitive detection in a low cost device. The present invention provides a process for the production of an optical filter comprising a dye-doped polydimethylsiloxane microfluidic layer and its incorporation into an integrated microfluidic device.

The first aspect of the invention provides a microfluidic device comprising a zone for receiving a sample, an excitation source and a detector, characterised in that a portion of the zone allows transmission of energy within a required wavelength range and prevents transmission of energy at all other wavelengths.

In particular, the portion of the zone is composed of a composition comprising polydimethylsiloxane and a dye, wherein said dye allows transmission of energy at a required wavelength range and prevents transmission of energy at all other wavelengths.

The microfluidic device is further provided wherein the zone allows transmission of energy at a required wavelength range and prevents transmission of energy at all other wavelengths. In particular, the zone is composed of a composition comprising polydimethylsiloxane and a dye, wherein said dye allows transmission of energy at a required wavelength range and prevents transmission of energy at all other wavelengths.

The zone for receiving the sample can therefore be provided as a monolithic structure, that is the zone is an integrated part of the microfluidic device.

The zone may be bounded by one or more walls. As discussed above the walls of the zone are an integrated part of the device. One or more of the walls or a portion of one or more of the walls can comprise the composition as defined above (i.e. a wall or a portion thereof can allow transmission of energy at a required wavelength range and prevent transmission of energy at all other wavelengths). Preferably all walls bounding the zone comprise the composition as described above. Preferably the wall or portion of wall comprising the composition as described above is arranged between the sample and the detector, between the sample and the light source or between the sample and the detector and between the sample and the light source. Preferably the microfluidic device comprises the polydimethylsiloxane composition as defined above. In particular, the polydimethylsiloxane composition can be used to produce part of a wall section, or a plate section sealing the zone.

It will be appreciated that the use of a composition comprising polydimethylsiloxane and a dye to produce at least part of a zone, avoids the need for a separate internal or external filter. Excitation of a sample within the zone will cause the sample to transmit energy. The presence of the polydimethylsiloxane composition in the microfluidic device and more particularly in the walls of the zone will allow transmission of energy at a required wavelength range while preventing transmission of energy at all other wavelengths. The microfluidic device and in particular the zone will act as a filter. It will be appreciated that the use of the polydimethylsiloxane composition in the manufacture of the microfluidic device allows the reduction and/or prevention of transmission of energy from a sample at undesirable wavelengths (i.e. it reduces the background noise from scattering of excitation light). The polydimethylsiloxane composition may further reduce ground noise from autofluorescence of a fluorescent and/or phosphorescent sample.

The device of the first aspect does not require an additional filter. Instead the incorporation of the composition into the microfluidic device of the first aspect allows the device to act as a short pass excitation light filter, a band pass filter, a long pass detection filter, or an absorbing filter to minimise scattering from the background to prevent direct or scattered light from reaching the detector.

The zone preferably has a depth of about 1 to 999 μm, preferably about 10 to about 500 μm, more preferably 20 to 100 μm. For optical detection it is sometimes useful to use deeper zones, for example 600-800 μm. The excitation source preferably comprises one or more light sources which can be integrated into the microfluidic device and/or which are external thereto. The excitation source may be a conventional lamp, a laser, a laser diode, an inorganic light emitting diode (LED) or an organic light emitting diode (OLED). The detector can be integrated into the microfluidic device and/or external thereto. The detector may be a photomuliplier tube or an inorganic or organic photodiode. The microfluidic device preferably comprises a substrate, for example a substrate chip, said zone being integrated into and/or supported thereon. The substrate may comprise the polydimethyl siloxane composition or any other material conventionally used in the art.

Any components of the device can be manufactured from the composition defined above. Preferably any components directly or indirectly in the pathway of the energy transmitted from the sample after excitation thereof are manufactured from the composition.

The device may comprise one or more layers of the polydimethylsiloxane composition. The layers can be 3 mm or less thick, preferably 2 mm or less thick, preferably 1 mm thick or less, preferably 100 micrometres thick or less, preferably 10 micrometres thick or less or preferably 1 micrometre thick or less. The layers can be stacked on top of each other and can be fused together reversibly by for example intrinsic adhesion or irreversibly by for example treatment with oxygen or air plasma or unfused. The use of such layers minimises the distance between the zones and a detector enabling high efficacy collection of the energy.

The microfluidic device therefore allows more sensitive detection of an analyte. The use of the device of the first aspect further allows superior light collection as the distance from the zone to the filter is minimised. The size of the device can also be kept to a minimum due to the reduction in additional components including additional external components.

The microfluidic device may be used for fluorescence detection of an analyte. The analyte may be a fluorescent and/or a phosphorescent analyte. Alternatively, the analyte may have been modified with a fluorophore and/or a phosphore to allow detection (for example, labelled protein for example in an immunoassay or labelled nucleic acid for example in a DNA hybridisation assay). In particular, an analyte comprising a fluorophore and/or a phosphore may be introduced into the microfluidic device and optionally modified (by a biological, physical or chemical process). Alternatively, the analyte may have been modified prior to its introduction into the microfluidic device. The fluorophore and/or phosphore undergoes detection in the device by excitation of the fluorophore and/or phosphore by a light source, the output of which is optionally sharpened using a short pass filter. The excited fluorophore and/or phosphore reemits light, said reemitted light being detected by a detector either integrated into the microfluidic device or external to the microfluidic device. The polydimethylsiloxane composition in the microfluidic device blocks excitation light at unwanted wavelengths, thereby enhancing the signal to noise ratio of the reemitted light and allowing more sensitive detection.

The microfluidic device may comprise one or more additional filters either internal and/or external to the microfluidic device. The additional filter can act as a short pass excitation light filter, a band pass filter, a long pass detection filter or an absorbing filter to minimising scattering from the background to prevent direct or scattered excitation light from reaching the detector. The microfluidic device preferably comprises an inorganic or organic semi-conductor based light source and/or an inorganic or organic semi-conductor based photodiode.

For the purposes of this invention, the dye is a low fluorescent, photostable compound which allows transmission of energy at a required wavelength range and prevents transmission of energy at all other wavelengths. Preferably, the dye is a lysochrome dye (i.e. a fat soluble dye). Examples of such lysochrome dyes include azo dyes which have undergone molecular rearrangement and are no longer able to ionise. It will be appreciated that selection of the dye will determine the wavelength range at which energy can be transmitted and the width of the wavelength range. The dye can therefore be used to tune the composition to allow transmission at the required wavelength range with the required or desired degree of selectivity and sensitivity.

Preferably, the dye comprises an aromatic system comprising one or more aromatic rings such as phenyl rings. More preferably, the dye comprises a conjugated aromatic system comprising two or more fused phenyl rings. The two or more fused rings may be directly fused to each other or may be fused to an intermediate moiety such as an unsaturated or partially saturated ring such as a cyclohexyl ring. In one feature of the first aspect of the invention, the dye comprises a compound of formula (I)

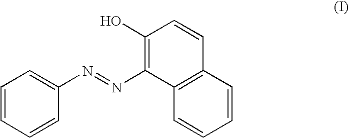

The compound of formula (I) may be substituted at one or more positions on the phenyl or napthalene ring with one or more of hydroxy, halo, C₁₋₄ alkyl.

For the purposes of the invention, the dye may comprise one or more selected from the Sudan dye family such as Sudan Blue II (Solvent Blue 35), Sudan Black B (Solvent Black), Sudan I (Solvent Yellow 14), Sudan II (Solvent Orange 7), Sudan III (Solvent Red 23) and Suden IV (Solvent Red 24), Solvent Blue 37, Solvent Blue 38, Solvent Blue 59 and Solvent Green 3 (Quinzarine Green 55). More preferably, the dye is selected from one or more of Sudan II,

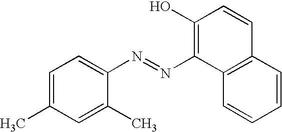

and/or Sudan IV

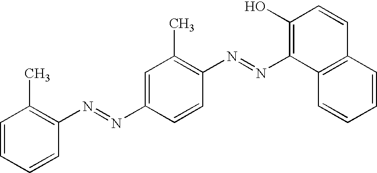

In an alternative or additional feature of the first aspect, the dye may be Sudan Blue II.

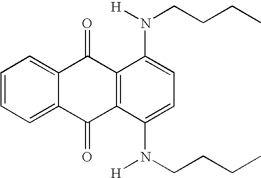

Alternatively the dye can be a porphyrin or a derivative thereof, pigment, colourant, or a nanoparticle such as CdS. It will be appreciated that two or more dyes can be incorporated into the polydimethylsiloxane composition to allow transmission of energy at two or more wavelengths (i.e. to create band pass filtration).

The dye may be provided in the composition in an admixture with the polydimethylsiloxane. Alternatively some or all of the dye may be reacted with polydimethylsiloxane monomers to form a co-polymer.

In particular, the microfluidic device comprises a composition comprising polydimethylsiloxane and Sudan II wherein said polydimethylsiloxane composition allows transmission at a wavelength of 570 nm or above and prevents transmission at a wavelength of below 520 nm.

The composition acts as a filter to allow transmission of energy, preferably light, at a wavelength or within a range of wavelengths. The transmission of energy at the specified wavelength or range of wavelengths is sufficient to allow detection of the transmitted energy. The composition further prevents transmission of energy (i.e. light) at all other wavelengths (i.e. at undesired or non-specified wavelengths). The composition does not necessarily prevent the transmission of 100% percent of the energy at an undesired wavelength. Instead the polydimethylsiloxane composition reduces the transmission of energy at an undesired wavelength sufficiently so that it does not interfere with the detection of energy at a desired wavelength or range of wavelengths.

The width of the range of wavelengths will depend on the dye or dyes used. In particular, transmission of energy will occur over a range of 100-300 nm.

The transition phase from transmission at a desired wavelength to non-transmission is preferably 100 nm or less, more preferably 50 nm or less. The composition of the invention allows broadband filtration for example the use of a composition comprising polydimethylsiloxane and Sudan Blue II in the manufacture of a microfluidic device allows transmission from 400 to 500 nm but prevents transmission at wavelengths less than 300 nm or greater than 550 nm. Alternatively the composition may allow narrow band filtration i.e. transmission over a wavelength range of 50 nm or less, preferably 20 nm or less, preferably 5 nm or less, preferably 1 nm or less.

It will be appreciated by a person skilled in the art that the incorporation of the dye into the composition determines the wavelength at which energy, for example light, can be transmitted through the polydimethylsiloxane composition. The composition can therefore be tuned according to the spectra of the excitation source (for example the excitation light source) and detection signal as required by the particular method of detection.

Preferably, the composition allows greater than 80% transmission of energy at a required wavelength, more preferably greater than 90% transmission of energy at a required wavelength, most preferably greater than 95% or above transmission of energy at a required wavelength. Furthermore, the composition preferably allows less than 10% transmission of energy at an undesired wavelength, preferably less than 5% transmission of energy at an undesired wavelength, preferably less than 2% or below transmission of energy at an undesired wavelength. Preferably 1% or below transmission of energy at an undesired wavelength, preferably 0.1% or below transmission of energy at an undesired wavelength, preferably 0.01% or below transmission at an undesired wavelength.

Preferably, the composition allows the transmission of light. Preferably the light is detectable as fluorescence or phosphorescence.

The microfluidic device can be produced at particularly low cost. Incorporation of the polydimethylsiloxane-dye composition into the device removes the need for separate conventional filters, which can be costly and time consuming to incorporate. In particular, interference filters require complex silicon based multilayer fabrication while gelatin filters require incorporation of a dye into liquid gelatin, coating of the gelatin-dye mixture onto glass, drying of the mixture, stripping of the mixture from the glass and coating with lacquer. These conventional filters then need to be incorporated into a microfabricated device.

The device of the present invention can be provided as a disposable device due to its low cost production. The production of the device is particularly cost effective when compared to the production of devices known in the art incorporating conventional filters, for example silicon based interference filters.

The use of the polydimethylsiloxane composition of the present invention allow the production of filters or microfluidic devices which exhibit low autofluorescence, negligible leaching with aqueous solutions and limited light induced degradation.

The spectral characteristics of the composition compare favourably with commercially available Schott glass long-pass filters, indicating that the invention allows the integration of high quality optical filters into the form of PDMS microfluidic chips. The use of the composition of the invention allows the production of filters which are robust in use, showing only slight degradation after extended illumination and negligible dye leaching after prolonged exposure to aqueous solutions. The present invention allows the provision of low cost high quality integrated filters and represents a key step towards the development of high-sensitivity disposable microfluidic devices for point-of-care diagnostics.

The second aspect of the invention relates to a process for the production of a composition comprising polydimethylsiloxane and a dye, comprising dissolving the dye in an apolar solvent, admixing the solubilised dye with a polydimethylsiloxane monomer and polymerising the polydimethylsiloxane monomer in the presence of the dye.

The polymerisation of the polydimethylsiloxane monomers in the presence of the dye allows precise dye concentration control and uniform dye incorporation.

The apolar solvent is preferably toluene, xylene or hexane. The process for producing the polydimethylsiloxane composition optionally includes adding a hardener to the polydimethylsiloxane monomers prior to polymerisation. In addition, the pre-polymerisation mixture may optionally be degassed prior to polymerisation.

The process of the second aspect allows the production of the composition in a reliable manner, thereby providing a material which can be used to manufacture a microfluidic device as disclosed in the first aspect.

The third aspect of the invention relates to a method of producing a microfluidic device as claimed in the first aspect of the invention. The composition comprises dissolving a dye in a non-polar solvent, admixing the solubilised dye and polydimethylsiloxane monomers, forming a predetermined object and curing the polymeric mixture. The device is preferably formed by the introduction of the solubilised dye and polydimethylsiloxane monomers into a mould, said final product being removed from the mould after curing.

The method may optionally comprise adding a hardener to the solubilised dye and polydimethylsiloxane monomers admixture. The device may be preferably formed by the introduction of the solubilised dye, the polydimethylsiloxane monomers and a hardener into a mould, said final product being removed from the mould after curing.

Curing of the polymeric mixture may be performed by baking the mixture in an oven at for example 95° for up to 2 hours or 65° C. for 4 hours, preferably 6 hours, more preferably 8 hours. Alternatively, the mixture can be left in the air to cure (for example for 24 hours or above at room temperature). The device can be prepared by casting or injection moulding. Where a portion of the zone for example one or more walls or a portion of one or more walls comprises the polydimethylsiloxane-dye composition the portion can be manufacture as discussed above and attached to a second polydimethylsiloxane portion which does not contain the dye such as a substrate plate.

The fourth aspect of the invention provides a composition comprising polydimethylsiloxane and a dye.

Preferably, the dye comprises an aromatic system comprising one or more aromatic rings such as phenyl rings. More preferably, the dye comprises a conjugated aromatic system comprising two or more fused phenyl rings. In one feature of the first aspect of the invention, the dye comprises a compound of formula (I)

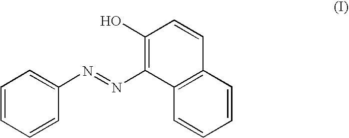

The compound of formula (I) may be substituted at one or more positions on the phenyl or napthalene ring with one or more of hydroxy, halo, C₁₋₄ alkyl.

For the purposes of the invention, the dye may comprise one or more selected from the Sudan dye family such as Sudan Blue II (Solvent Blue 35), Sudan Black B (Solvent Black), Sudan I (Solvent Yellow 14), Sudan II (Solvent Orange 7), Sudan III (Solvent Red 23), Sudan IV (Solvent Red 24), Solvent Blue 37, Solvent Blue 38, Solvent Blue 59 and Solvent Green 3 (Quinizarine Green 55). More preferably, the dye is selected from one or more of Sudan II,

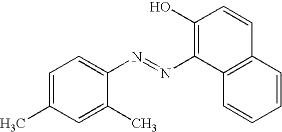

and/or Sudan IV

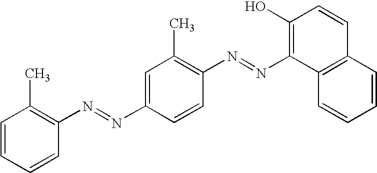

In an alternative or additional feature of the first aspect, the dye may be Sudan Blue (II).

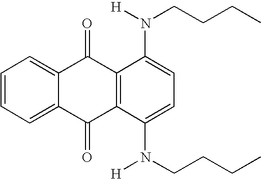

Alternatively the dye can be a porphyrin or a derivative thereof, colourant, pigment or a nanoparticle such as CdS. It will be appreciated that two or more dyes can be incorporated into the polydimethylsiloxane composition to allow transmission of energy at two or more wavelengths (i.e. to create band pass filtration).

The dyes of the fourth aspect are soluble in apolar solvents such as toluene, xylene or hexane. The dyes further exhibit a low tendency for aggregation.

The composition can be used to produce optical components for incorporation into a device for example a device of the first aspect of the invention. In particular, the composition can be incorporated into or used to produce a filter, lens, prism, microlens, reaction zone, sensor, substrate plate, etc.

The fifth aspect relates to a diagnostic test comprising introducing a sample into a microfluidic device of the first aspect of the invention optionally modifying the sample by a chemical, physical or biological process, exciting the sample (for example by illumination) and detecting the emitted energy. In particular, the test of the fifth aspect allows fluorescence or phosphorescence detection, preferably fluorescence detection of an analyte. The analyte may be a fluorescent and/or phosphorescent analyte. Alternatively the analyte may be modified by a fluorophore or a phosphore. For example an analyte may be a fluorescently or phosphorescently labelled protein (for example in an immunoassay) or nucleic acid (for example in a DNA hybridisation assay). The analyte comprising a fluorophore and/or a phosphore is introduced into the microfluidic device and optionally modified. Alternatively a non-fluorophore or phosphorescent analyte is introduced into the microfluidic device and is modified such that is fluoresces and/or phosphoresces. The fluorophore and/or phosphore is excited by a light source and the light re-emitted from the fluorophore and/or phosphore is detected.

The fluorophore or phosphore is preferably a labelled protein, nucleic acid or a portion or derivative thereof.

The diagnostic test preferably comprises a test for detecting the presence or absence of an analyte, preferably by fluorescence and/or phosphorescence.

The sixth aspect of the invention relates to a kit for carrying out a diagnostic test comprising a microfluidic device of the first aspect of the invention and instructions relating to the diagnostic test to be carried out.

All preferred features of each of the aspects of the invention apply to all other aspects mutatis mutandis.

The invention may be put into practice in various ways and a number of specific embodiments will be described by way of example to illustrate the invention with reference to the accompanying drawings, in which:

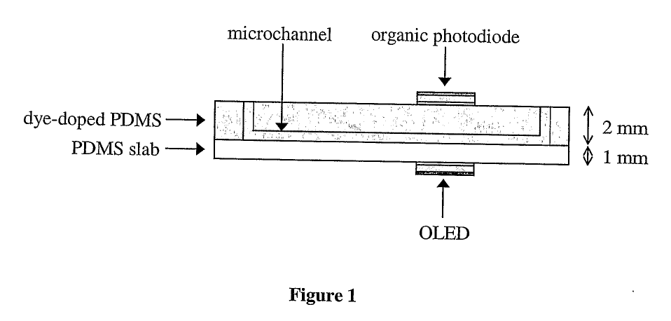

FIG. 1 shows a typical configuration for using the dye-doped PDMS filters. A monolithic microfluidic filter layer is sealed against an unstructured PDMS slab. Fluorescent dyes are pumped through the microchannel inlet and excited with an OLED light source. In a collinear geometry, fluorescence emitted from the dye is detected with an organic photodiode through the doped PDMS filter layer;

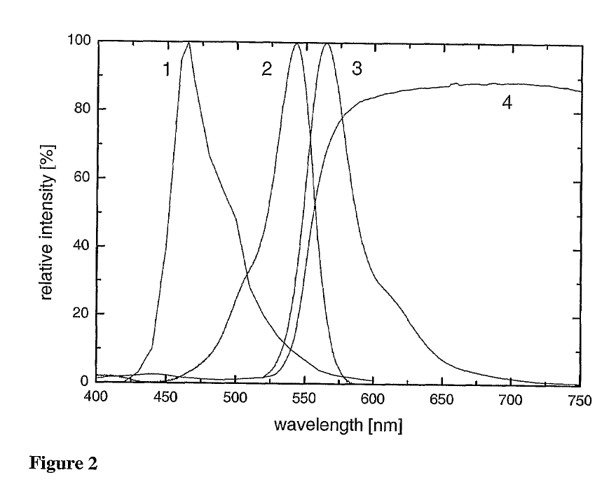

FIG. 2 shows an emission spectrum of a blue OLED (1) and the overlap with excitation (2) and emission spectra (3) of the model fluorophore Rhodamine B. The Sudan II doped optical filter layer (4) acts as a long-pass filter, effectively blocking OLED emission from reaching the detector;

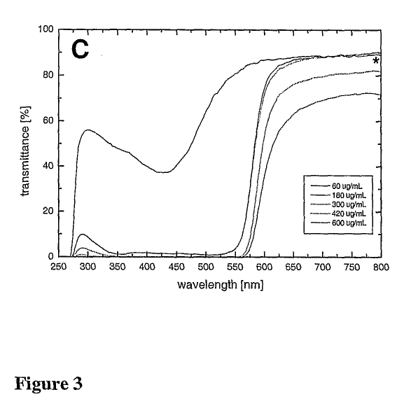

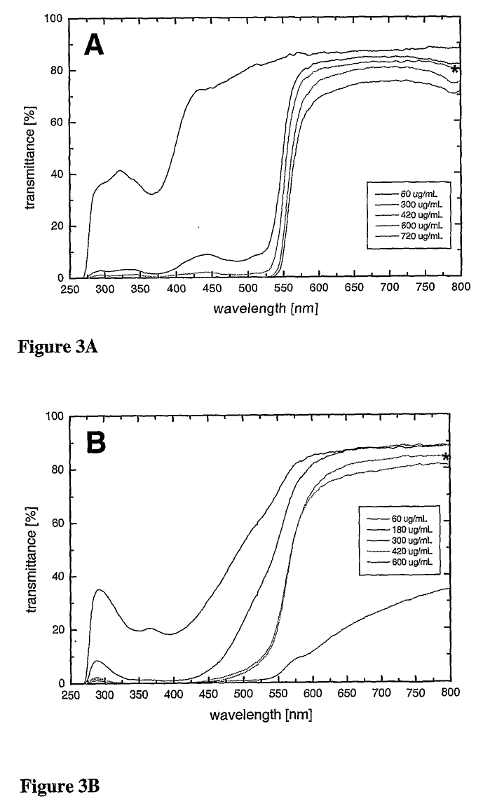

FIG. 3 shows the transmission characteristics of 3-mm-thick PDMS layers doped with varying concentrations of Sudan II (A), Sudan III (B) and Sudan IV (C) dyes. Asterisks denote spectra for optimized conditions;

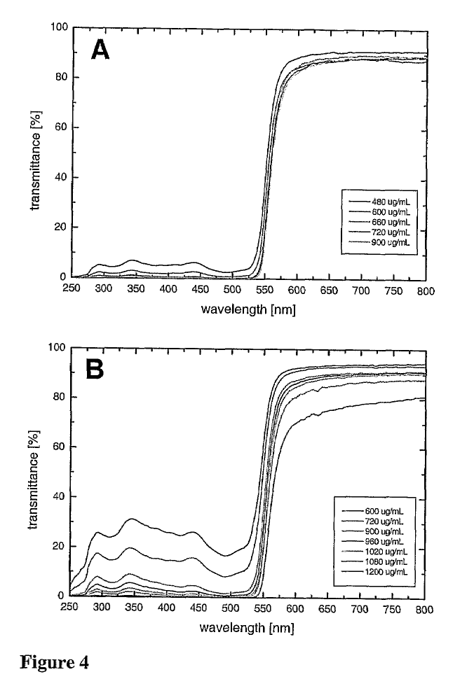

FIG. 4 shows the transmission characteristics for 2-mm-thick (A) and 1-mm-thick (B) thin-film PDMS layers doped with Sudan II;

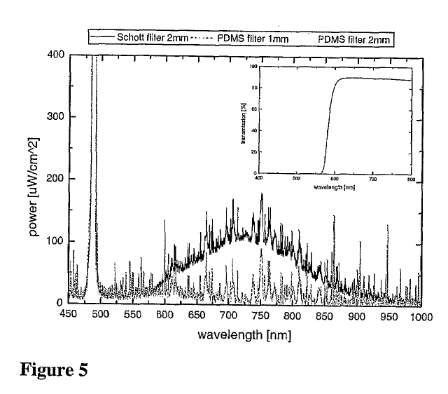

FIG. 5 shows autofluorescence measurements for a 2-mm-thick commercial Schott filter and 1-mm and 2-mm-thick PDMS layers doped with 1200 and 900 micro g/mL Sudan II, respectively. A 200 mW Ar Ion laser was used as excitation light source. Fluorescence emission was measured inside an integration sphere with a CCD spectrometer. Inset shows the transmission spectrum of the 582-nm cut-on Schott filter;

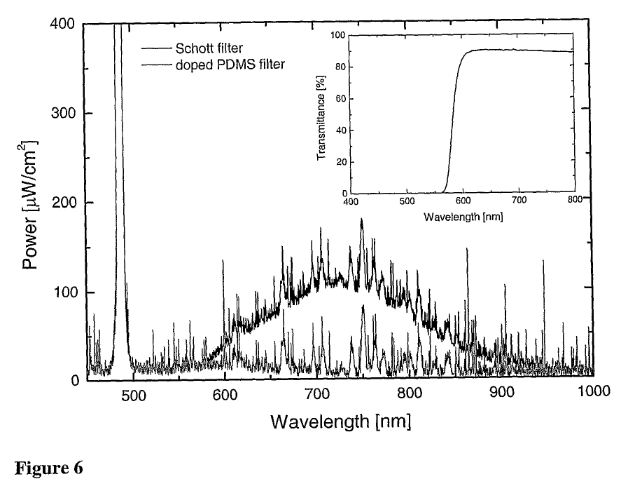

FIG. 6 shows autofluorescence detection results for a 2 mm commercial Schott filter and 2 mm PDMS layers doped with 900 μg/mL Sudan II. A 488 nm Ar Ion laser was used as the excitation source. Fluorescence emission was measured inside an integrating sphere with a high-sensitivity CCD spectrometer. Inset shows the transmission spectrum of the Schott filter with a cut-on point at 583 nm. The dye-loading of the PDMS filter was selected to approximately match the optical density of the Schott filter for the same filter thickness;

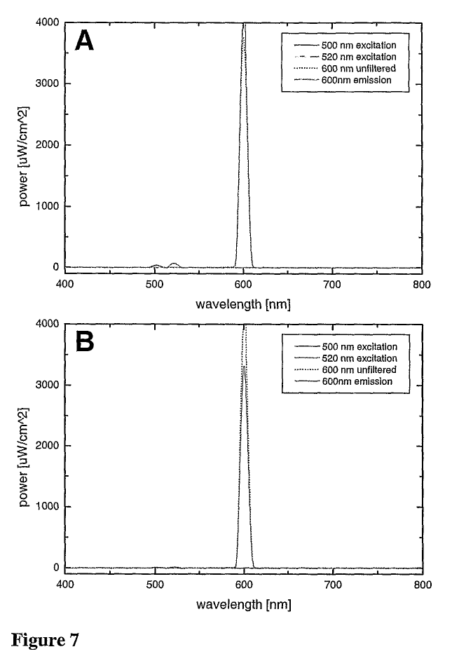

FIG. 7 illustrates the selective blocking properties of the optimised 1-mm-thick (A) and 2-mm-thick (B) Sudan II doped PDMS layers. For excitation monochromatic light at 500 and 520 nm was employed, overlapping with the absorption band of the model fluorophore Rhodamine B. Both wavelengths are effectively blocked by the filter layer. Longer wavelength emission from the fluorophore, however, is transmitted. This is illustrated with the monochromatic light at 600 nm. The transmitted light is only fractionally lower than for undoped PDMS (see dotted line);

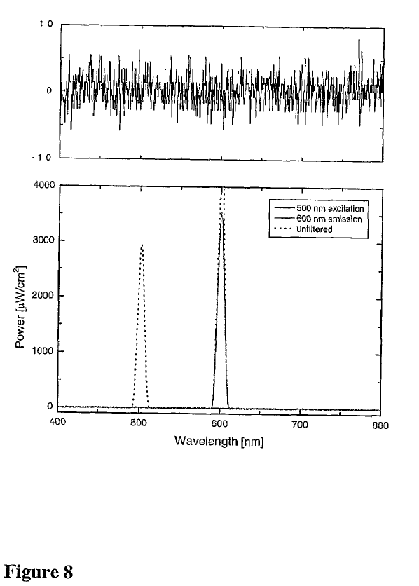

FIG. 8 shows the blocking properties of a 1 mm ˜OD4 Sudan II filter, measured using the collinear configuration described herein. The lower plot indicates that the 500 nm light is strongly attenuated by the dye-doped PDMS while the 600 nm light is transmitted at a comparable level to that for the undoped PDMS. The upper plot shows the 500 nm data for the dye-doped PDMS on a greatly magnified scale. There is no detectable signal over the full spectral range, indicating that the intensity of the transmitted light and the intensity of filter autofluorescence are both below the noise floor of the spectrometer;

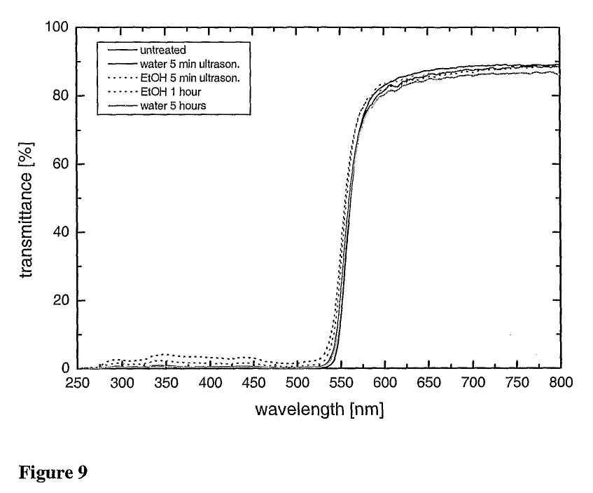

FIG. 9 shows the filter stability against chemical solvents. For chemical stability testing, filters were ultrasonicated in water or ethanol. Presented data is for 2-mm-thick PDMS layers doped with 600 micro g/mL Sudan II;

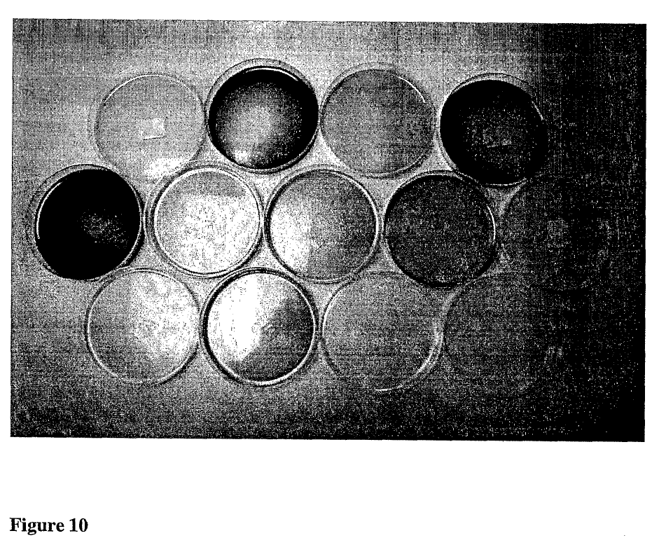

FIG. 10 shows different dyes doped into PDMS forming colour plate in a peridish;

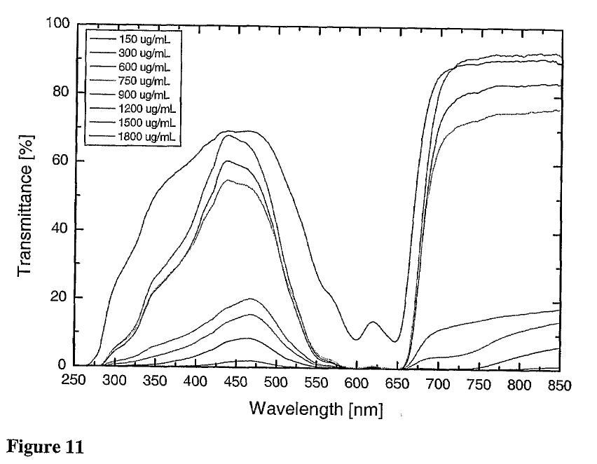

FIG. 11 shows transmission spectra of 2-mm-thick Sudan Blue (II) doped PDMS at different doping levels;

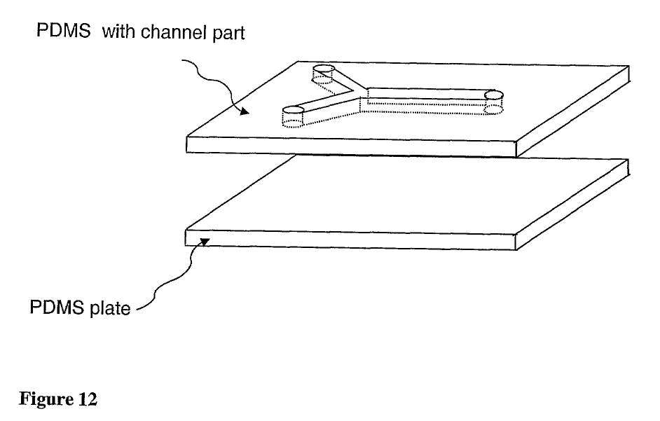

FIG. 12 shows a diagram of a microfluidic chip wherein the colour filter can be made into the microfluidic chip directly, as one part of the channel section and/or the plate section for sealing the chip;

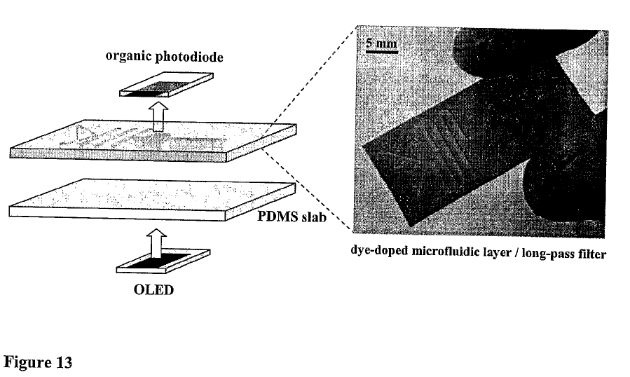

FIG. 13 shows a schematic of monolithically integrated optical long-pass filter. The structured dye-doped PDMS layer serves concurrently as a microfluidic circuit and optical long-pass filter. Enclosed microchannels are obtained by sealing the doped layer against a second non-doped PDMS slab. For a collinear detection geometry the excitation source and detector are positioned below and above the assembled microchip. The inset to FIG. 13 shows an image of a Sudan II doped PDMS filter with 800-micrometre-wide and 800 micrometre-deep microchannels;

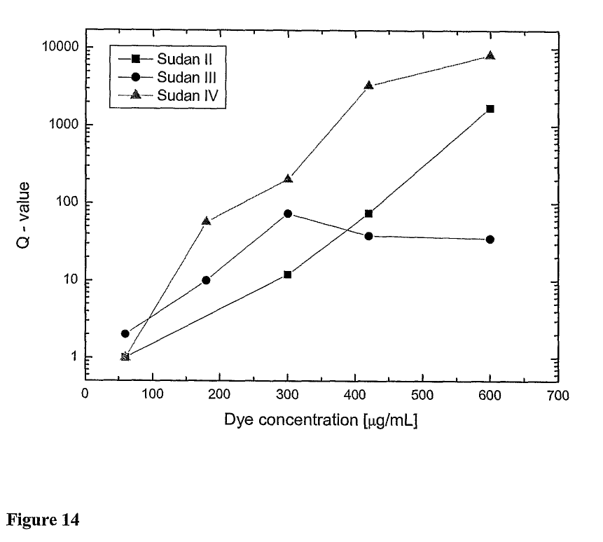

FIG. 14 shows the figure of merit Q for 3 mm PDMS layers doped with varying concentrations of Sudan II, Sudan III and Sudan IV. Q corresponds to the ratio of filter transmission at wavelengths above and below the cut-on point. For Sudan II (cut-on 550 nm) transmission values at 600 and 500 nm were used. For Sudan III (cut-on 560 nm) and Sudan IV (cut-on 580 nm), transmissions at 650 and 470 nm, and 630 and 530 nm were used, respectively; and

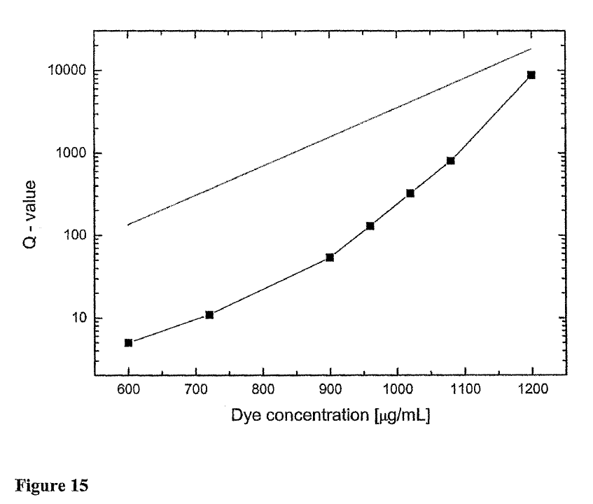

FIG. 15 shows the figure of merit Q for 1 mm PDMS layers doped with varying concentrations of Sudan II. The dotted line corresponds to the theoretical limit based on the extinction coefficient of Sudan II above and below the cut-on point.

The present invention will now be illustrated by reference to one or more of the following non-limiting examples.

### EXAMPLES

### Optical Filter Fabrication

Optical long-pass filters were fabricated by pre-polymerization doping of polydimethylesiloxane (PDMS). Sudan II, III, IV and Sudan Blue II dyes (Sigma-Aldrich, Gillingham, UK) were dissolved in 1 mL toluene and than added to 16.5 mL of PDMS monomer and hardener pre-mixed at a ratio of 10:1 v/v (Sylgard 184 Silicone Elastomer kit, Dow Corning, Coventry, UK). Vigorous manual mixing was continued until a uniform PDMS colouring was obtained. The doped PDMS was then poured into plastic Petri dishes for fabrication of unstructured filters or over an SU-8 master for molding of the monolithically integrated microfluidic/filter layer. The two-level SU-8 master was fabricated on a silicon substrate at the Centre of Integrated Photonics (Ipswich, UK) using standard SU-8 processing protocols. Filter thickness was controlled via the poured PDMS volume, with a standard filter thickness of 3 mm. For filter performance optimization the thickness was later reduced to 2 mm and 1 mm, with adjusted dye load. PDMS curing was performed at room temperature for 48 hours. For high Sudan II dye loads, a 4 hour 65° C. curing step was added.

Filters were prepared at various thicknesses (1-3 mm) and dye-loadings (60-1800 μg/mL). The highest dye loads necessitated an increase of the toluene content in order to achieve full dissolution of the dye molecules and uniform dispersion in the final matrix. However, excessively high solvent content resulted in incomplete polymerization even after prolonged curing. The overall solvent content in the doped PDMS was thus limited to below 10 v/v/-%. For high dye loads, a 4 hour 65° C. curing step was added to ensure complete hardening of the PDMS.

Transmittance of the dye-doped PDMS filters was measured on a UV-VIS Spectrometer V-560 (Jasco, Great Dunmow, UK) equipped with a customized thin-film holder. Spectra were baseline corrected and referenced against air. Typical scans were performed from 250 to 850 nm at 400 nm/min with 2 nm line width. Autofluorescence of the Sudan filter range was measured with a 200 mW Ar Ion laser excitation source and a USB 2000 CCD spectrometer (Ocean Optics, Duiven, The Netherlands) using an integration sphere.

Filter stability against continuous light exposure was tested by illuminating the filters with UV light (200 W mercury arc lamp) or storing the filters on a laboratory bench and exposing them to ambient light for 4 weeks. Chemical stability was tested by filters immersed in water or ethanol being ultrasonicated at 33 kHz (300 W) for 5 min, 10 min, 1 hour, 3 hours and 5 hours (Sonomatic S1000, Langford Electronics, Birmingham, UK). The resulting optical properties were then compared to those of untreated PDMS filters.

### Microfluidic Chip Fabrication

For the fabrication of monolithically integrated microfluidic/filter layers, the Sudan II doped PDMS monomer/hardener solution was poured over an SU-8 master prior to curing. The SU-8 master was fabricated on a silicon substrate using standard SU-8 processing protocols (Centre for Integrated Photonics, Ipswich, UK). The cured PDMS layer, shown in FIG. 13, was then peeled off and sealed against an unstructured slab of PDMS to form enclosed microchannels. The fidelity of feature replication was unaffected by the dye doping, allowing for facile fabrication of micron-sized features. Bulk PDMS properties such as elasticity, wettability and bonding characteristics were also unaffected. The favourable properties of the PDMS were therefore retained even after dye-doping.

### Fluorescence Detection Set-Up

For use of the monolithically integrated microfluidic/filter layers in on-chip fluorescence detection, access holes are punched at the channel ends with a glass Pasteur pipette, resulting in ˜2-mm-diameter holes, followed by reversible sealing against a 1-mm-thick unstructured PDMS slab. Rhodamine B solutions of different concentration are then hydrodynamically pumped through the microchip inlet(s). In a collinear detection geometry, a blue OLED and an organic photodiode can be used as the excitation source and detector, respectively (FIG. 1). The blue OLED emission ranges from 420 to 650 nm, with peak emission at 465 nm. Rhodamine B has excitation and emission maxima of 540 and 570 nm, respectively. The employed optimized dye-doped PDMS filters employed exhibit <2% transmittance below 520 nm and 90% transmittance above 570 nm (FIG. 2). This enables effective blocking of excitation light while fluoresence emission from Rhodamine B is transmitted. Without doping of the PDMS layer, the photodiode would be saturated by direct excitation light, preventing the detection of low intensity fluorescence light. Responsivity of the organic photodiode is typically from 400 to 800 nm.

A more rigorous measurement of filter autofluorescence may be obtained using an integrating sphere—a hollow sphere that has its inner surface coated with a diffusely reflecting material. When a light-source—in this case an autofluorescent filter—is placed inside an ideal integrating sphere the light is redistributed isotropically over the sphere's interior-surface, irrespective of the angular distribution of the emission. Hence, if N_{Ω} photons of a given wavelength are detected over a solid angle Ω, the total number N of emitted photons is given by N=k_{λ}N_{Ω}(4π/Ω) where k_{λ} is a wavelength dependent constant that accounts for reflection losses in the sphere. The use of an integrating sphere therefore enables the total number of photons emitted via autofluorescence to be determined from a measurement at a single location on the sphere wall, obviating the need to map out the full angular distribution of the emission. The PDMS filters were placed inside an 18 cm diameter sphere (Labsphere) and illuminated with the 2 mm diameter beam output from a 200 mW 488 nm Ar Ion laser (43 Series Ar Ion laser, Melles Griot) entering through a small inlet port. The emitted light was detected using a fibre-optic coupled CCD spectrometer (USB 2000 CCD spectrometer with 600 μm diameter fiber, Ocean Optics) inserted into a second port, oriented at 90 degrees relative to the laser beam. The autofluorescence characteristics of the Sudan II PDMS filters were measured as well as those of a commercially available Schott glass long-pass filter with a cut-on point at 583 nm. The Schott filter showed a small amount of autofluorescence in the range 570 to 950 nm but no fluorescence was measurable from the PDMS filter, confirming the high quality of the Sudan II/PDMS filters (FIG. 6). Since the autofluorescence was too weak to register on the CCD spectrometer, the external photoluminescence quantum efficiency of the filter could not be calculated. However, based on the sensitivity of the system, it was certainly less than 0.01%.

Optimization of Chromophore Incorporation into the PDMS Host Matrix.

For comparative purposes, post-polymerization staining with polar dyes as previously reported by Whitesides was performed with Rhodamine B and Rhodamine 640. While in both cases staining was observed after immersion in aqueous dye solution for 48 hours, a cross-sectional cut revealed heavy staining only near the surfaces. This is consistent with a diffusional staining process. Attempts to increase the dye intake by plasma treatment of the PDMS surface to enhance wettability, or by increasing the monomer/hardener ratio from 10:1 to 50:1 v/v to enlarge pore size were unsuccessful.

Pre-polymerization doping was tested by adding a chromophore to the monomer/hardener mix prior to curing. For the PDMS host matrix, this required apolar chromophores since the Sylgard monomer is dissolved in hexane and xylene. Addition of polar dyes dissolved in water or ethanol resulted in emulsive non-uniform chromophore incorporation and incomplete polymerization. In contrast, apolar Sudan dyes dissolved in small volumes of hexane or toluene and thoroughly mixed with PDMS monomer/hardener were uniformly incorporated. The applied solvent volume was minimized such that the overall solvent content in the doped PDMS was below 10 v/v-%. Higher solvent contents often resulted in incomplete polymerization even after prolonged curing.

The optical properties of Sudan doped PDMS layers after curing are depicted in FIG. 3. The cut-on point for Sudan II based long-pass filters is 550 nm, corresponding to the wavelength at 50% peak transmission. For Sudan II it can be seen that dye concentrations below 300 micro g/mL resulted in high transmission above 570 nm, but insufficient blocking below 520 nm (FIG. 3a). An increase of dye concentration enhanced low-wavelength blocking but also reduced high-wavelength transmission. The optimum dye concentration was 420 micro g/mL, yielding ˜1%-transmission below 520 nm and up to 85%-transmission above 570 nm. Sudan III dye incorporation resulted in generally higher transmission above 570 nm, but a broader transition phase between low-wavelength blocking and high-wavelength transmission (FIG. 3b). The cut-on point was 560 nm. Optimal results were obtained for 300 micro g/mL resulting in <0.5%-transmission below 450 nm and 85%-transmission above 620 nm. Sudan IV doped filters with 580 nm cut-on points typically yielded excellent low-wavelength blocking, a moderately sharp transition phase and good high-wavelength transmission (FIG. 3c). Optimal results were obtained for 300 micro g/mL with <0.5%-transmission below 500 nm and up to 90%-transmission above 620 nm. For other applications using a strong excitation light source, it may be beneficial to use filters with higher dye load and enhanced low wavelength blocking.

Since in fluorescence detection a sharp cut-on between excitation light blocking and transmission of emitted fluorescence is crucial, Sudan II doped layers with typical transition phases <50 nm were selected for further optimization. Thin-film filters are beneficial for efficient collection of fluorophore emission and as such the effect of filter thickness was subsequently investigated. FIG. 4 depicts the changes associated with reducing the filter thickness for Sudan II doped PDMS layers. Doping 2-mm-thick layers with 600 micro g/mL Sudan II resulted in <1%-transmittance at 500 nm and ˜90%-transmittance above 570 nm (FIG. 4a). This is an improvement over the 3-mm-thick layers yielding ˜85% high-wavelength transmittance. Increasing the dye load to 720 and 900 micro g/mL yielded enhanced low-wavelength blocking with 500 nm transmittance reduced to <0.5 and 0.05%, corresponding to optical densities of 2.3 and 3.3, respectively (OD=−log(% T/100). Decreasing the layer thickness to 1 mm without adjusting the dye load resulted in improved high-wavelength transmission but ineffective low wavelength attenuation (FIG. 4b). When the dye load was increased to 1080 and 1200 micro g/mL, however, excellent low wavelength blocking was achieved with a 500 nm transmittance of 0.1% (OD 3) and 0.007% (OD 4.2), respectively. This represents state-of-the-art attenuation levels and enables the use of powerful light sources such as lasers, laser diodes, LEDs and OLEDs. A further increase of the dye load up to 2400 micro g/mL resulted in reduced high wavelength transmission without further enhancement of low wavelength blocking. We believe that the filter blocking properties are predominantly determined by the total amount of incorporated dye. Consequently thinner films require higher dye loads for optimum results. However, this trend is limited by dye solubility, i.e. excessive dye loads can result in non-uniform suspension-type dye incorporation. Dye loads ≧2400 micro g/mL induce particle agglomeration, even with increased solvent content.

Autofluoresence of the filters was tested using a 200 mW Ar Ion laser, an integration sphere and a CCD spectrometer equipped with an optical fibre. The barium sulfate coated integration sphere diffuses all light uniformly throughout the sphere and enhances collection of isotropically emitted fluorescence. The PDMS filters were placed inside the sphere and illuminated with the 2 mm diam. laser beam entering through a sphere orifice. Detection of any emitted autofluorescence originating from the filter was performed with the CCD spectrometer fibre inserted into a second orifice, oriented at 90 degrees relative to the laser beam. The autofluorescence of optimised 1-mm and 2-mm-thick PDMS layers doped with 1200 and 900 mg/mL Sudan II, respectively, and of a commercial 2-mm-thick Schott filter are depicted in FIG. 5. It can be seen that while the Schott filter shows considerable autofluorescence from ˜570 to 950 nm, the Sudan II doped PDMS filters exhibit no measurable autofluorescence. This confirms that the presented method is capable of fabricating optical-grade filters and meets industrial standards.

To demonstrate the excellent selective blocking characteristics of the presented filters they were illuminated with monochromatic light mimicking excitation and emission wavelengths typically used for the model fluorophore Rhodamine B (FIG. 7) It can be seen that for both the 1-mm-thick and 2-mm-thick doped PDMS layers the excitation light at 500 and 520 nm is effectively blocked while the longer wavelength fluorescence emission is transmitted to similar levels as for undoped PDMS. These characteristics in conjunction with the low autofluoresence represent excellent long-pass filtering properties.

For the optimized 2 mm Sudan II doped filters, stability against chemical solvents and prolonged light exposure was subsequently investigated, which are important parameter for industrial applications (FIG. 9). It can be seen that ultrasonication in water for 5 min resulted in only negligible changes with low-wavelength transmission increased by 0.15% and high-wavelength transmission reduced by <2%. Ultrasonicating the filters in ethanol for 5 min, such as commonly used in microchip cleaning protocols resulted in similar negligible changes. Soaking of the doped filters in ethanol for 1 hour resulted in a transmission change of ˜1.5% while soaking in ethanol for 5 hours resulted in a ˜2% drop of high wavelength transmission, with negligible changes to the low wavelength blocking. These changes are attributed to limited dye leaching which is stronger for ethanol (due to higher Sudan dye solubility) and is enhanced by ultrasonication induced cavitation. For use in disposable devices this solvent stability is clearly satisfactory. Prolonged exposure of the doped filters to ambient light in the laboratory revealed a slight deterioration of low wavelength blocking after 4 weeks, which we attribute to minute dye bleaching (500 nm transmittance increased from <0.1 to 0.5%). Similarly, exposure to a 200 W UV lamp for up to 1 hour and a 200 mW Ar Ion laser for up to 30 min caused only minimal changes. We consider this light stability appropriate, particularly for the anticipated use in disposable diagnostic system which are routinely packaged in air tight aluminum foil wrapping.

For shortpass or bandpass filter applications, Sudan Blue II was dissolved in toluene to form different concentration solutions with 15 microg/ml, 10 microg/ml, 5 microg/ml, respectively. It was necessary to determine that the dyes were fully dissolved. One volume liquid solution was mixed with 10 volumes of PDMS monomer and one volume hardener, the mixture was then poured into a pre-shaped mould to form defined shape components (i.e. plate, chip, or lens). The mixture was degassed to remove air bubbles trapped in the mixture before curing the monomer. Curing was carried out by leaving the mixture in the air at the room temperature. Preshaped rubber like flexible PDMS components can be obtained.

The transmission spectra of Sudan Blue II doped into PDMS formed plate with 2 mm thickness were taken by UV-VIS spectrophotometer shown in FIG. 11. The different concentrations of Sudan Blue II solution mixed with pre-polymer PDMS were used in order to optimise the transmission spectra. From the spectra, a suitable concentration for the 2 mm thick PDMS plate can be used for colour filtering to transmit in the blue or red region spectrum and block from 550 nm to 650 nm.

Filters can therefore be made from the PDMS composition and can be used for fluorescence detection. The composition can be introduced directly into a microfluidic device either as one part of a channel section or as a plate section for sealing the chip (as shown in FIG. 12). The device can act as a filter for excitation light or detection.

The ideal longpass filter should provide excellent attenuation and transmission either side of the cut-on, and we therefore used the convenient figure of merit Q=T(λₜᵣₐₙₛₘᵢₜ)/T(λ_{block}) to assess the filter performance, where T(λ) is the percentage transmission at a wavelength λ, and λₜᵣₐₙₛₘᵢₜ and λ_{block} correspond to convenient wavelengths either side of the filter cut-on. Q corresponds to the expected improvement in sensitivity when clear PDMS is replaced by the dye-doped PDMS if the following criteria are met: (i) the spectrum of the light source lies entirely in the attenuation region of the long-pass filter; (ii) the analyte emission spectrum lies entirely in the transmissive region of the long-pass filter; (iii) the limit-of-detection is determined by the background due to the excitation light rather than detector noise or other electronic noise; and (iv) the autofluorescence from the filter is negligible. If these criteria are satisfied then the optimal filter performance is obtained at the dye loading that maximises Q.

In preliminary tests, to assess the relative performance of the three dye molecules, 3-mm-thick filters were fabricated using Sudan II, Sudan III and Sudan IV dye loadings in the range 60 to 720 μg/mL. The concentration dependence of the Q-values for the three dyes are shown in FIG. 3. The Q-values for Sudan II and Sudan IV increase rapidly with dye-loading, consistent with improved short wavelength attenuation as the dye concentration increases. In the case of Sudan III, the Q-value at first increases exponentially with dye loading but rapidly reaches a peak at 300 μg/mL, and then decreases. This reduction in filter performance is attributable to aggregation of the dye molecules since, at dye-loadings above 300 μg/mL, dye molecule particulates become visible in the doped PDMS layers, signifying non-uniform dye incorporation. Sudan II and IV are both promising filter materials but Sudan III is insufficiently soluble in toluene to achieve adequate filter performance. It is probable that an alternative solvent choice would permit high quality Sudan III filters to be fabricated.

A sharp roll-on between attenuation and transmission is crucial in fluorescence detection, so Sudan II filters, which have a typical roll-on transition width <50 nm, were selected for further optimization. In order to ensure efficient collection of fluorophore emission in a microfluidic environment, the distance between the fluorophore and detector should be kept small, and subsequent tests were therefore conducted on 1 mm filters, which is typical of the substrate thicknesses employed in microfluidic devices. In FIG. 4, we show the Q-values obtained using Sudan II filters with dye concentrations in the range 600 to 1200 μg/mL, where the upper limit corresponds to the maximum dye loading that could be achieved without noticeable dye aggregation. The Q-value increases sharply with concentration over the full concentration range, reaching a maximum value of ˜8800 at 1200 μg/mL. If the four criteria discussed above are satisfied, this represents a potential increase in sensitivity of almost four orders of magnitude. The Q-value increases super-exponentially over the concentration range investigated which is somewhat surprising since, if the dye were incorporated uniformly, Q would be related to the dye concentration by the following exponential relationship

$$Q = \frac{{T}_{long}}{{T}_{short}} ∝ \frac{{e}^{- c d {ɛ}_{long}}}{{e}^{- c d {ɛ}_{short}}} ∝ {e}^{c d ( {ɛ}_{short} - {ɛ}_{long} )}$$

where c is the dye concentration, d is the filter thickness, and ε_{long} and εₛₕₒᵣₜ represent the extinction coefficients of the dye molecule at the long and short wavelengths respectively. The extinction coefficients of Sudan II were measured in a 100 μM solution in toluene, yielding values of 24280 and 170 M⁻¹cm⁻¹ at 500 and 600 nm respectively. This corresponds to a maximum theoretical Q-value of 18260 for 1200 μg/mL (˜4 mM) Sudan II in a 1-mm layer, substantially higher than the value actually obtained in our PDMS filters. The ideal Q value as a function of dye concentration is shown as a dotted line in FIG. 4 and, as expected, the observed data lies below the calculated optimum over the entire range. The lower non-exponential nature of the experimental data indicates that the dye is incorporated non-uniformly within the host matrix, which results in sub-optimal blocking characteristics. Microscopic studies of the polymer blend phase structure at different dye loadings are currently under way, with a view to obtaining improved dispersion of the dye and hence Q-values closer to the theoretical maximum. At high dye loadings the deviation between expected and measured Q is a factor of 2 only.

### SUMMARY

High quality monolithically integrated disposable PDMS based microfluidic layers were fabricated with optical long-pass filter characteristics. The use of Sudan II and Sudan IV dyes showed good solubility and yielded high quality filters with cut-on wavelengths of 550 and 580 nm respectively. For instance, 3-mm-thick filters with relatively low dye-loadings of 600 μg/mL yielded short wavelength optical densities of 3.3 and 4.3 for Sudan II and Sudan IV, respectively. Using Sudan II as a test dye, high quality 1 mm PDMS layers were fabricated with dye-loadings up to 1200 μg/mL. The resultant filters had excellent optical characteristics, e.g. <0.01%-transmission at 500 nm and >80%-transmission above 570 nm. Importantly, the filters showed negligible autofluorescence, allowing them to be effectively employed in microchip-based fluorescence detection. The filters proved robust in use, undergoing only negligible leaching in aqueous solution and marginal photodegradation. Patterning of the PDMS was unaffected by the dye doping, allowing for the fabrication of coloured substrates that serve concurrently as channel medium and optical filter.

1. A microfluidic device comprising a zone for receiving a sample, an excitation source and a detector, characterised in that a portion of the zone comprises a low fluorescent, lysochrome dye which comprises an aromatic system, and allows transmission of energy at a required wavelength range and prevents transmission of energy at all other wavelengths.

2. The microfluidic device as claimed in claim 1 wherein the portion of the zone is composed of a composition comprising polydimethylsiloxane and a low fluorescent, lysochrome dye which comprises an aromatic system, wherein said dye allows transmission of energy at a required wavelength range and prevents transmission of energy at all other wavelengths.

3. The microfluidic device as claimed in claim 1 wherein the zone allows transmission of energy at a required wavelength range and prevents transmission of energy at all other wavelengths.

4. The microfluidic device as claimed in claim 3 wherein the zone is composed of a composition comprising polydimethylsiloxane and a low fluorescent, lysochrome dye which comprises an aromatic system, wherein said dye allows transmission of energy at a required wavelength range and prevents transmission of energy at all other wavelengths.

5. The microfluidic device as claimed in claim 1 wherein the zone is bounded by one or more walls wherein said one or more walls or a portion of said one or more walls comprises a composition comprising polydimethylsiloxane and a low fluorescent, lysochrome dye which comprises an aromatic system, wherein said dye allows transmission of energy at a required wavelength and prevents transmission of energy at all other wavelengths.

6. The microfluidic device as claimed in claim 1 wherein the excitation source comprises one or more light sources

7. The microfluidic device as claimed in claim 1 wherein the dye comprises a compound of formula (I) 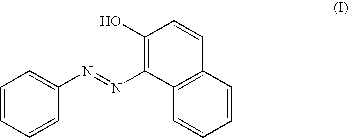
optionally substituted at one or more positions on the phenyl or napthalene ring with one or more of hydroxy, halo, C₁₋₄ alkyl.

8. The microfluidic device as claimed in claim 7 wherein the dye is selected from the Sudan dye family.

9. The microfluidic device as claimed in claim 7 wherein the dye is one or more of Sudan II, 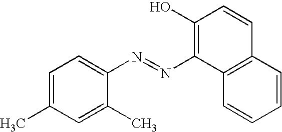
Sudan IV 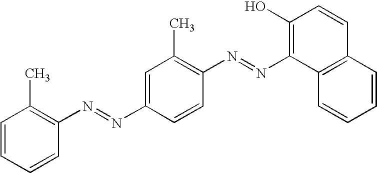
or Sudan Blue (II)

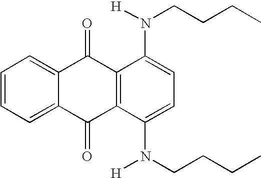

10. The microfluidic device as claimed in claim 2 wherein the composition comprises polydimethylsiloxane and Sudan II wherein said polydimethylsiloxane composition allows transmission at a wavelength of 570 nm or above and prevents transmission at a wavelength of below 520 nm.

11. A process for the production of the composition comprising polydimethylsiloxane and a dye, comprising dissolving the dye in an apolar solvent, admixing the solubilised dye with a polydimethylsiloxane monomer and polymerising the polydimethylsiloxane monomer in the presence of the dye.

12. The process as claimed in claim 11 wherein the apolar solvent is toluene, xylene or hexane.

13. The process as claimed in claim 11 wherein a hardener is added to the polydimethylsiloxane monomer prior to polymerisation.

14. A method of producing a microfluidic device as claimed in claim 1 comprising dissolving a dye in a non-polar solvent, admixing the solubilised dye and polydimethylsiloxane monomer, forming a predetermined object and curing the polymeric mixture.

15. The method as claimed in claim 14 wherein the device is formed by the introduction of the solubilised dye and polydimethylsiloxane monomers into a mould, said final product being removed from the mould after curing.

16. The method as claimed in claim 14 wherein the solubilised dye and polydimethylsiloxane monomer are admixed in the presence of a hardener.

17. A composition comprising polydimethylsiloxane and a dye, wherein said dye comprises a compound of formula (I) 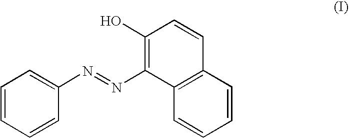
optionally substituted at one or more positions on the phenyl or napthalene ring with one or more of hydroxy, halo, C₁₋₄ alkyl.

18. A composition as claimed in claim 17 wherein said dye is one or more selected from the Sudan dye family.

19. A composition as claimed in claim 18 wherein the dye is one or more of Sudan II, 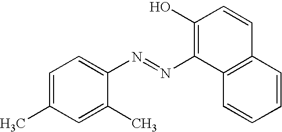
or Sudan IV 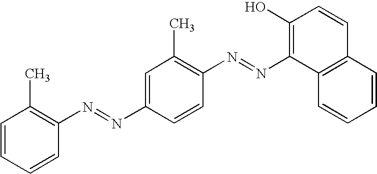
or Sudan Blue II 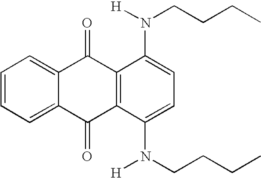
or a porphyrin or a derivative thereof, colourant, pigment of a nanoparticle.

20. A diagnostic test comprising introducing a sample into a microfluidic device as claimed in claim 1 exciting the sample and detecting the emitted energy.

21. A diagnostic test as claimed in claim 20 further comprising modification of the sample prior to excitation by chemical, physical or biological modification.

22. A diagnostic test as claimed in claim 20 wherein the emitted energy is generated by fluorescence or phosphorescence.

23. A diagnostic test as claimed in claim 20 comprising introducing a sample comprising a fluorophore or phosphore into the microfluidic device, exciting the sample with a light source and detecting the light re-emitted from the sample.

24. A kit for carrying out a diagnostic test comprising a microfluidic device as claimed in claim 1 and instructions relating to the diagnostic test to be carried out.

25-29. (canceled)
